(19) United States
(12) Patent Application Publication
(10) Pub. No.: US 2020/0155057 A1
(43) Pub. Date: May 21, 2020

(54) AUTOMATIC DETECTION OF ASPIRATION-PENETRATION USING SWALLOWING ACCELEROMETRY SIGNALS

(71) Applicant: HOLLAND BLOORVIEW KIDS REHABILITATION HOSPITAL, Toronto, Ontario (CA)

(72) Inventors: Ali-Akbar Samadani, Cambridge, MA (US); Tom Chau, Toronto, Ontario (CA)

(21) Appl. No.: 16/634,290

(22) PCT Filed: Jul. 26, 2018
(86) PCT No.: PCT/CA2018/050909

§ 371 Date: Jan. 27, 2020

Related U.S. Application Data
(60) Provisional application No. 62/537,767, filed on Jul. 27, 2017.

(51) Int. Cl. A61B 5/00

(52) U.S. Cl. CPC A61B 5/4205; A61B 5/6822; A61B 5/7203; A61B 2562/0204; A61B 5/7267; A61B 2562/0219; A61B 5/726

(57) ABSTRACT

A method can use dual-axis accelerometry signals obtained during a swallow to classify the swallow as a normal swallow or as an impaired swallow (e.g., an aspiration-penetration). The method can include representing the dual-axis accelerometry signals as meta-features, comparing the salient time and frequency meta-features, identified by regularized binomial logistic regression with elastic net penalty performed on the time and frequency meta-features in a known training data set, with a preset linear discriminant classifier constructed based on the salient meta-features, and classifying the swallow as a normal swallow or a possibly impaired swallow, based on the comparing. Preferably a processing module operatively connected to the sensor performs the processing of the dual-axis accelerometry signals and also automatically classifies the swallow.

### BACKGROUND

The present disclosure generally relates to methods and devices for classifying a swallowing event. More specifically, the present disclosure relates to methods and devices that distinguish between a swallow with aspiration-penetration and a swallow without aspiration-penetration using a classifier based on dual-axis accelerometry meta-features most salient to detecting swallowing aspiration-penetration.

Dysphagia is characterized by impaired involuntary motor control of swallowing process and can cause “penetration” which is the entry of foreign material into the airway. The airway invasion can be accompanied by “aspiration” in which the foreign material enters the lungs and can lead to serious health risks.

The three phases of swallowing activity are oral, pharyngeal and esophageal. The pharyngeal phase is typically compromised in patients with dysphagia. The impaired pharyngeal phase of swallowing in dysphagia is a prevalent health condition (38% of the population above 65 years) and may result in prandial aspiration (entry of food into the airway) and/or pharyngeal residues, which in turn can pose serious health risks such as aspiration pneumonia, malnutrition, dehydration, and even death. Swallowing aspiration can be silent (i.e., without any overt signs of swallowing difficulty such as cough), especially in children with dysphagia and patients with acute stroke, rendering detection via clinical perceptual judgement difficult.

The current gold standard for tracking swallowing activities is videofluoroscopy that enables clinicians to monitor barium-infused foodstuff during swallowing via moving x-ray images. However, the videofluoroscopy swallowing study (VFSS) cannot be done routinely due to the expensive procedure and the need for specialized personnel, as well as the excessive amount of harmful radiations. Another invasive assessment is the flexible endoscopic evaluation of swallowing, which also requires trained personnel and entails an expensive procedure. Non-invasive alternatives for swallow monitoring include surface electromyography, pulse oximetry, cervical auscultation (listening to the breath sounds near the larynx) and swallowing accelerometry.

Despite introduction of different non-invasive approaches, a reliable bedside detection of swallowing abnormalities remains a challenging task. For example, a recent systematic review of cervical auscultation studies suggests that the reliability of the approach is insufficient and it can not be used as a stand-alone instrument to diagnose dysphagia. Lagarde, Marloes L J and Kamalski, Digna M A and van den Engel-Hoek, Lenie, “The reliability and validity of cervical auscultation in the diagnosis of dysphagia: A systematic review,” Clinical Rehabilitation 30(2):1-9 (March 2015). Furthermore, perceptual clinical screening of dysphagia has been shown to lack agreement between different speech-language pathologists, possibly due to the subjective nature of the judgement as well as the presence of variety of environmental artifacts.

Over the past two decades, researchers have reported on various swallowing screening tools among which those driven by swallowing sounds are the most popular ones. Swallowing sounds are either captured acoustically using a microphone or mechanically using an accelerometer placed on the patient's neck measuring cervical epidermal vibrations. Reports on discriminative analysis of swallowing auscultation signals vary in terms of the screening tool used, target swallowing problem (aspiration, penetration, pharyngeal residue), sample size, patient population and medical conditions, and validation approach, which makes a direct comparison between these studies virtually impossible.

Swallowing accelerometry harnesses the hyoid and laryngeal movements during swallowing activities, which are manifested as epidermal vibrations measurable at the neck by an accelerometer. Vibrations in both the anterior-posterior (A-P) and superior-inferior (S-I) anatomical directions are found to contain distinct information about the underlying swallowing activities.

Previous swallowing accelerometry studies reported on a small sample size, and the swallowing samples in these studies were all collected by the researchers at a single site. Furthermore, swallowing screening studies often target a single population of patients (e.g., post-stroke patients, pediatric population with physical disabilities). The high-level of heterogeneity of the previous studies preclude meaningful comparison between them and a reliable assessment of proposed swallowing screening tools. Moreover, many of the previous works on swallowing accelerometry employ single-axis accelerometers in the A-P direction.

### SUMMARY

The present inventors surprisingly discovered a particular automatic framework for detecting aspiration-penetration based on dual-axis (A-P and S-I) accelerometry signals captured at the patient's neck during swallowing. The framework represents the accelerometry signals in terms of time and frequency meta-features, preceded by signal preprocessing and conditioning. The meta-features representation can then be used in regularized binomial logistic regression with elastic net penalty to identify features most salient to detecting swallowing aspiration-penetration. The identified salient features can then be exploited to devise a classifier based on linear discriminant analysis that receives as input a set of bolus accelerometry signals from a participant and returns associated labels at both bolus and participant levels, indicating the presence or absence of aspiration-penetration for the participant.

As detailed herein, the performance of the framework was evaluated using a large dataset of swallowing activities (up to 298 participants) collected from 8 different sites. Participants were asked to consume boluses of thin liquid barium and nectar-thick liquid barium (mild consistency), and their swallowing activities were captured simultaneously using videofluoroscopy and a dual-axis accelerometer attached to the participant's neck. The discriminative framework achieves bolus-level sensitivity/specificity/area under the ROC curve (AUC) rates of greater than 80%/60%/0.8, respectively, in detecting swallowing aspiration-penetration in both the thin and mild consistencies.

Accordingly, in a general embodiment, the present disclosure provides a method to classify a swallow. The method comprises: receiving, on a processing module, dual-axis accelerometry signals obtained during the swallow by a sensor positioned externally on an anterior-posterior (A-P) axis and a superior-inferior axis (S-I) of the throat of a subject; representing the dual-axis accelerometry signals as meta-features, the processing module performs the representing; identifying a subset of the meta-features using regularized binomial logistic regression with elastic net penalty, the processing module performs the identifying; and using the subset of the meta-features for determination of a linear discriminant classifier, the processing module performs the determination.

In an embodiment, the method comprises converting the dual-axis accelerometry signals from bivariate bolus signals to univariate bolus signals which are represented as a set of meta-features.

In an embodiment, the meta-features comprise time-frequency characteristics of the accelerometry signals.

In an embodiment, the meta-features comprise one or more channel-specific head-motion features. The meta-features can comprise, for each of the one or more channel-specific head-motion features, a ratio of the channel-specific head-motion feature for the A-P axis to the corresponding channel-specific head-motion feature for the S-I axis.

In an embodiment, the method comprises tuning the linear discriminant classifier by performing cross-validation to identify salient meta-features by regularized binomial logistic regression with elastic net penalty and to optimize at least one of sensitivity or specificity of the linear discriminant classifier. The linear discriminant classifier can comprise a bolus-level threshold and a participant-level threshold, and the tuning of the linear discriminant classifier can comprise tuning the bolus-level threshold and the participant-level threshold separately from each other.

In an embodiment, the method further comprises: receiving, on the processing module, a set of bolus accelerometry signals; applying the linear discriminant classifier to the set of bolus accelerometry signals; and providing on the processing module or a device operatively connected to the processing module an indication whether the set of bolus accelerometry signals comprises an aspiration-penetration, the indication based on the applying of the linear discriminant classifier to the set of meta-features representing the bolus accelerometry signals.

In another embodiment, the present disclosure provides a method to classify a swallow, the method comprising: receiving, on a processing module, dual-axis accelerometry signals obtained during the swallow by a sensor positioned externally on an anterior-posterior (A-P) axis and a superior-inferior axis (S-I) of the throat of a subject; representing the dual-axis accelerometry signals as meta-features, the processing module performs the representing; comparing the meta-features, identified by regularized binomial logistic regression with elastic net penalty performed on the time and frequency meta-features in a known training data set, with a preset linear discriminant classifier constructed on salient time and frequency meta-features in a known training data set, the processing module performs the comparing, the processing module performs the comparing; and classifying the swallow as a normal swallow or an aspiration-penetration, the processing module performs the classifying based on the comparing.

In an embodiment, the method comprises converting the dual-axis accelerometry signals from bivariate bolus signals to univariate bolus signals that each are an inner product signal, and representing the inner product signals in terms of a subset of the identified meta-features according to the bolus consistency.

In an embodiment, the meta-features comprise time-frequency characteristics of the accelerometry signals.

In an embodiment, the meta-features comprise one or more channel-specific head-motion features. The meta-features can comprise, for each of the one or more channel-specific head-motion features, a ratio of the channel-specific head-motion feature for the A-P axis to the corresponding channel-specific head-motion feature for the S-I axis.

In an embodiment, the method comprises tuning the linear discriminant classifier by performing cross-validation to identify salient meta-features by regularized binomial logistic regression with elastic net penalty and to optimize at least one of sensitivity or specificity of the linear discriminant classifier.

In another embodiment, the present disclosure provides an apparatus for quantifying swallowing function. The apparatus comprises: a sensor configured to be positioned on the throat of a patient and acquire vibrational data representing swallowing activity and associated with an anterior-posterior axis and a superior-inferior axis; and a processing module operatively connected to the sensor and configured to (i) represent the vibrational data as salient meta-features identified by regularized binomial logistic regression with elastic net penalty performed on time and frequency meta-features in a known training data set, (ii) compare the salient meta-features with a preset linear discriminant classifier constructed using the time and frequency meta-features in the known training data set; and (iii) classify the swallow as a normal swallow or an aspiration-penetration, based on comparison of the salient meta-features with the preset linear discriminant classifier.

In an embodiment, the apparatus comprises an output component selected from a display, a speaker, and a combination thereof, the processing module configured to use the output component to indicate the classification of the swallow visually and/or audibly.

In an embodiment, the processing module is operatively connected to the sensor by at least one of a wired connection or a wireless connection.

In an embodiment, the processing module is configured to convert the vibrational data from bivariate bolus signals to univariate bolus signals that each are an inner product signal, and a subset of the meta-features are selected according to the bolus consistency.

In another embodiment, the present disclosure provides a method of treating dysphagia in a patient. The method comprises: positioning a sensor externally on the throat of the patient, the sensor acquiring vibrational data representing swallowing activity and associated with at least one axis selected from the group consisting of an anterior-posterior axis and a superior-inferior axis, the sensor operatively connected to a processing module configured to (i) represent the vibrational data as salient meta-features identified by regularized binomial logistic regression with elastic net penalty performed on time and frequency meta-features in a known training data set; (ii) compare the salient meta-features with a preset linear discriminant classifier trained on the known training data set; and (iii) classify the swallow as a normal swallow or an aspiration-penetration, based on comparison of the salient meta-features with the preset linear discriminant classifier.

In an embodiment, the adjusting of the feeding is selected from the group consisting of: changing a consistency of the feeding, changing a type of food in the feeding, changing a size of a portion of the feeding administered to the patient, changing a frequency at which portions of the feeding are administered to the patient, and combinations thereof.

An advantage of one or more embodiments provided by the present disclosure is to overcome drawbacks of known techniques for swallowing impairment detection.

Another advantage of one or more embodiments provided by the present disclosure is to provide an economically viable and accurate bedside swallow screening technology.

A further advantage of one or more embodiments provided by the present disclosure is to detect impaired swallowing activities to thereby facilitate prompt and effective swallow management intervention in high-risk populations.

Yet another advantage of one or more embodiments provided by the present disclosure is to improve the reliability of the swallowing accelerometry modality of detecting swallowing aspiration-penetration.

Another advantage of one or more embodiments provided by the present disclosure is to exploit swallowing accelerometry signals in A-P and S-I directions to detect swallowing anomalies, in particular by focusing on the aspiration-penetration problem and presenting a framework for discriminating between safe and unsafe (airway invasion at or below the true vocal folds) using dual-axis swallowing accelerometry signals.

A further advantage of one or more embodiments provided by the present disclosure is to implement more effective dysphagia intervention that reduces the health risks associated with aspiration-penetration.

Additional features and advantages are described herein, and will be apparent from, the following Detailed Description and the Figures.

### BRIEF DESCRIPTION OF THE FIGURES

FIG. 1 is diagram showing the axes of acceleration in the anterior-posterior and superior-inferior directions.

FIG. 2 is a schematic diagram of an embodiment of a swallowing impairment detection device in operation.

FIG. 3 is a schematic diagram of an embodiment of a method of discriminating swallowing aspiration-penetration.

FIG. 4 is a schematic diagram of the approach employed in the experimental example disclosed herein.

FIG. 5 is a table showing the number of patients and boluses used in the experimental example disclosed herein.

FIG. 6 is a schematic diagram of a cross-validation test used in the experimental example disclosed herein.

FIG. 7 is a table of classification rates (%) for thin bolus test sets in the safe versus unsafe classification in the experimental example disclosed herein; in each cross-validation run, salient features were selected and classifier threshold was tuned using the training set only (296 participants in this experiment).

FIG. 8 is a table of classification rates (%) for mild bolus test sets in the safe versus unsafe classification in the experimental example disclosed herein; in each cross-validation run, salient features were selected and classifier threshold was tuned using the training set only (298 participants in this experiment).

FIGS. 9A-9D are graphs showing distributions of tuned classifier thresholds at bolus- and participant-levels in the 1,000 runs of hold-out cross-validation test for thin and mild consistencies in the experimental example disclosed herein.

FIG. 10 is a table showing the top six selected features using elastic net in 1,000 runs of the hold-out cross-validation in the experimental example disclosed herein.

### DETAILED DESCRIPTION

As used in this disclosure and the appended claims, the singular forms “a,” “an” and “the” include plural referents unless the context clearly dictates otherwise. As used herein, “about” is understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, all numerical ranges herein should be understood to include all integers, whole or fractions, within the range.

The words “comprise,” “comprises” and “comprising” are to be interpreted inclusively rather than exclusively. Likewise, the terms “include,” “including” and “or” should all be construed to be inclusive, unless such a construction is clearly prohibited from the context. A disclosure of a device “comprising” several components does not require that the components are physically attached to each other in all embodiments.

Nevertheless, the devices disclosed herein may lack any element that is not specifically disclosed. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified.

The term “and/or” used in the context of “X and/or Y” should be interpreted as “X,” or “Y,” or “X and Y.” Where used herein, the terms “example” and “such as,” particularly when followed by a listing of terms, are merely exemplary and illustrative and should not be deemed to be exclusive or comprehensive. Any embodiment disclosed herein can be combined with any other embodiment disclosed herein unless explicitly stated otherwise.

As used herein, a “bolus” is a single sip or mouthful or a food or beverage. As used herein, “aspiration” is entry of food or drink into the trachea (windpipe) and lungs and can occur during swallowing and/or after swallowing (post-deglutitive aspiration). Post-deglutitive aspiration generally occurs as a result of pharyngeal residue that remains in the pharynx after swallowing.

An aspect of the present disclosure is a method of processing dual-axis accelerometry signals to classify one or more swallowing events. A non-limiting example of such a method classifies each of the one or more swallowing events as a swallow with aspiration-penetration or a swallow without aspiration-penetration. Another aspect of the present disclosure is a device that implements one or more steps of the method.

In an embodiment, the method can further comprise classifying the patient as having safe swallowing or unsafe swallowing. For example, a patient can be classified as having unsafe swallowing if the one or more swallowing events comprise an amount or percentage of aspiration-penetration events that exceeds a threshold. In such an embodiment, the threshold can be zero such that the presence of any aspiration-penetration events classifies the patient as having unsafe swallowing. Of course, in other such embodiments, the threshold can be greater than zero.

In some embodiments, the method and the device can be employed in the apparatus and/or the method for detecting aspiration disclosed in U.S. Pat. No. 7,749,177 to Chau et al., the method and/or the system of segmentation and time duration analysis of dual-axis swallowing accelerometry signals disclosed in U.S. Pat. No. 8,267,875 to Chau et al., the system and/or the method for detecting swallowing activity disclosed in U.S. Pat. No. 9,138,171 to Chau et al., or the method and/or the device for swallowing impairment detection disclosed in U.S. Patent App. Publ. No. 2014/0228714 to Chau et al., each of which is incorporated herein by reference in its entirety.

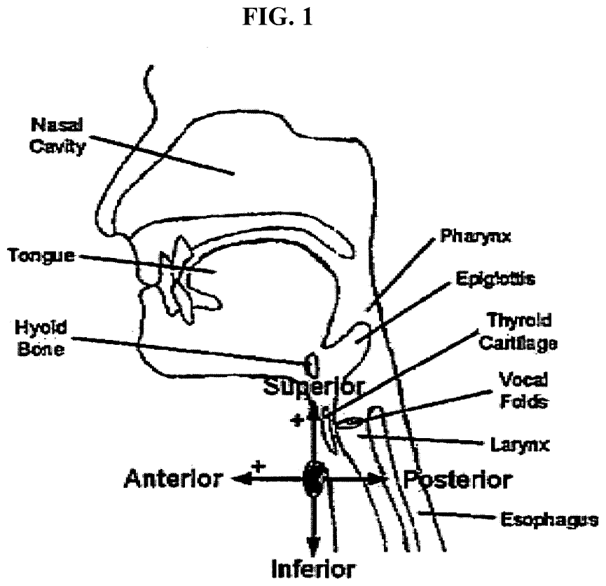

As discussed in greater detail hereafter, the device may include a sensor configured to produce signals indicating swallowing activities (e.g., a dual axis accelerometer). The sensor may be positioned externally on the neck of a human, preferably anterior to the cricoid cartilage of the neck. A variety of means may be applied to position the sensor and to hold the sensor in such position, for example double-sided tape. Preferably the positioning of the sensor is such that the axes of acceleration are aligned to the anterior-posterior and super-inferior directions, as shown in FIG. 1.

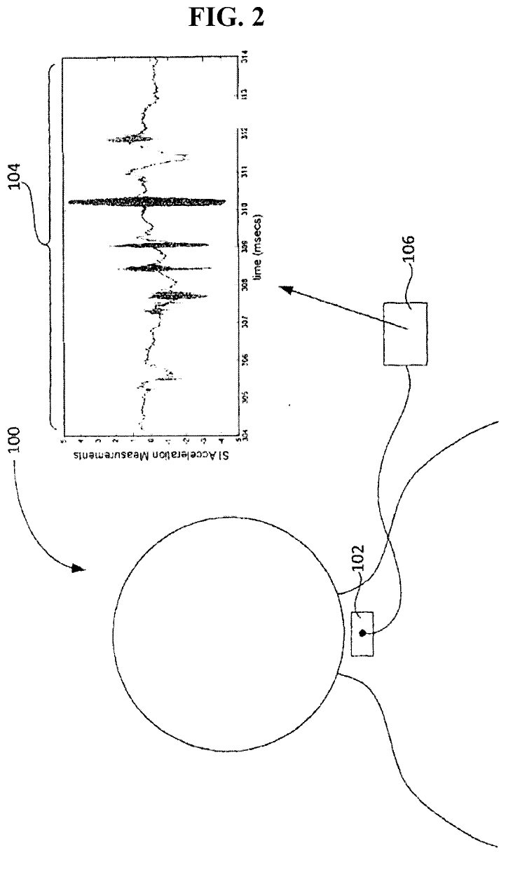

FIG. 2 generally illustrates a non-limiting example of a device 100 for use in swallowing impairment detection. The device 100 can comprise a sensor 102 (e.g., a dual axis accelerometer) to be attached in a throat area of a candidate for acquiring dual axis accelerometry data and/or signals during swallowing, for example illustrative S-I acceleration signal 104. Accelerometry data may include, but is not limited to, throat vibration signals acquired along the anterior-posterior axis (A-P) and/or the superior-inferior axis (S-I). The sensor 102 can be any accelerometer known to one of skill in this art, for example a single axis accelerometer (which can be rotated on the patient to obtain dual-axis vibrational data) such as an EMT 25-C single axis accelerometer or a dual axis accelerometer such as an ADXL322 or ADXL327 dual axis accelerometer, and the present disclosure is not limited to a specific embodiment of the sensor 102.

The sensor 102 can be operatively coupled to a processing module 106 configured to process the acquired data for swallowing impairment detection, for example aspiration-penetration detection and/or detection of other swallowing impairments such as swallowing inefficiencies. The processing module 106 can be a distinctly implemented device operatively coupled to the sensor 102 for communication of data thereto, for example, by one or more data communication media such as wires, cables, optical fibers, and the like and/or by one or more wireless data transfer protocols. In some embodiments, the processing module 106 may be implemented integrally with the sensor 102.

Generally, the processing of the dual-axis accelerometry signals comprises representation of the signals in time-frequency meta-features and then swallowing event classification based on the time-frequency meta-features. In applying this approach, the swallowing events may be effectively classified as a normal swallowing event or a potentially impaired swallowing events (e.g., unsafe and/or inefficient). Preferably the classification is automatic such that no user input is needed for the dual-axis accelerometry signals to be processed and used for classification of the swallow.

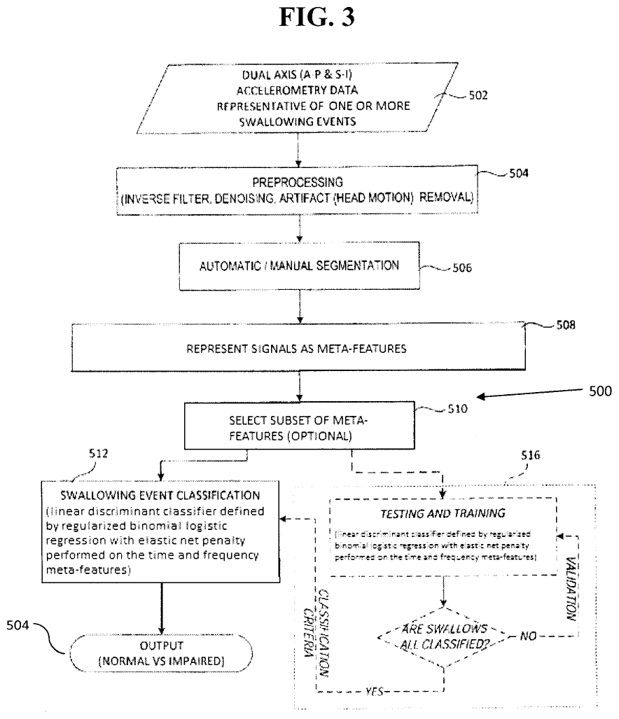

FIG. 3 illustrates a non-limiting embodiment of a method 500 for classifying a swallowing event. At Step 502, dual-axis accelerometry data for both the S-I axis and the A-P axis is acquired or provided for one or more swallowing events, for example dual-axis accelerometry data from the sensor 102.

At Step 504, the dual-axis accelerometry data can optionally be processed to condition the accelerometry data and thus facilitate further processing thereof. For example, the dual-axis accelerometry data may be filtered, denoised, and/or processed for signal artifact removal (“preprocessed data”). In an embodiment, the dual-axis accelerometry data is subjected to an inverse filter, which may include various low-pass, band-pass and/or high-pass filters, followed by signal amplification. A denoising subroutine can then applied to the inverse filtered data, preferably processing signal wavelets and iterating to find a minimum mean square error.

In an embodiment, the preprocessing may comprise a subroutine for the removal of movement artifacts from the data, for example, in relation to head movement by the patient. Additionally or alternatively, other signal artifacts, such as vocalization and blood flow, may be removed from the dual-axis accelerometry data. Nevertheless, the method 500 is not limited to a specific embodiment of the preprocessing of the accelerometry data, and the preprocessing may comprise any known method for filtering, denoising and/or removing signal artifacts.

At Step 506, the accelerometry data (either raw or preprocessed) can then be automatically or manually segmented into distinct swallowing events. Preferably the accelerometry data is automatically segmented. In an embodiment, the segmentation is automatic and energy-based. In another embodiment, the accelerometry data is automatically segmented as disclosed in U.S. Pat. No. 8,267,875 to Chau et al., the entirety of which is incorporated herein by reference as noted above. For example, the automatic segmentation can comprise applying fuzzy c-means optimization to the data determine the time boundaries for each of the swallowing and non-swallowing segments. Additionally or alternatively, manual segmentation may be applied, for example by visual inspection of the data. The method 500 is not limited to a specific process of segmentation, and the process of segmentation can be any segmentation process known to one skilled in this art.

At Step 508, meta-feature based representation of the signals is performed. Preferably the dual-axis accelerometry data (e.g., bivariate bolus signals that have been preprocessed and/or normalized) are converted to univariate signals using the windowed inner-product of their A-P and S-I channels with a predetermined window size (e.g., 750 samples) and a predetermined amount of overlap between successive windows (e.g., 90% overlap). The resulting univariate bolus signals (referred to as “inner-product signals” hereafter) can then be represented in terms of meta-features. The meta-feature representation of bolus signals can then be used as the input along with respective labels in subsequent feature-selection and/or classification.

At Step 510 (which is optional), a subset of the meta-features may be selected for classification, for example based on the previous analysis of similar extracted feature sets derived during classifier training and/or calibration. Such preselected feature components/levels can then be used to train the classifier for subsequent classifications. Ultimately, these preselected meta-features can be used in characterizing the classification criteria for subsequent classifications. For example, selection of the meta-features can be implemented using regularized binomial logistic regression with elastic net penalty on the classifier training data set and the selected features can be used to distinguish safe swallows from potentially unsafe swallows. The extraction of the selected meta-features from new test data can be compared to preset classification criteria established as a function of these same selected meta-features as previously extracted and reduced from an adequate training data set, to classify the new test data as representative of a safe swallow or unsafe swallow.

Accordingly, where the device has been configured to operate from a reduced feature set, such as described above, this reduced feature set will be characterized by a predefined feature subset or feature reduction criteria that resulted from the previous implementation of a feature reduction technique on the classifier training data set. Newly acquired data will thus proceed through the pre-processing and segmentation steps described above (Steps 504 and 506), the various swallowing events so identified then processed for feature extraction at Step 508 (e.g. full feature set), and those features corresponding with the preselected subset retained at step 510 for classification at Step 512.

Preferably, the meta-feature selection comprises using a regularized binomial logistic regression with elastic net penalty to identify meta-features most salient to detecting swallowing aspiration-penetration. For example, the meta-features preferably comprise at least one of the group consisting of (i) entropy rate of the inner product signal, (ii) length of the bolus, (iii) Hjorth complexity of the derivative of the inner product signal, (iv) energy of S-I head motion, (v) Hjorth mobility of AP head motion, (vi) relative energy of A-P head motion to S-I head motion, (vii) Hjorth mobility of the derivative of the inner product signal, and (viii) Hurst exponent of the inner product signal [ADD OTHER SALIENT META-FEATUES]. In such an embodiment, the meta-features can be any number of these features (i)-(viii), for example one, two, three, four, five, six, seven or even all eight of these features [revise sentence to conform to additions to previous sentence].

At Step 512, feature classification can be implemented. Preferably a linear discriminant analysis is used as a Bayesian classifier to detect aspiration-penetration events. The input into the classifier can be a set of bolus accelerometry signals from the patient (e.g., the segmented raw or preprocessed data), and the output from the classifier can be associated labels at bolus and/or participant level indicating the presence or absence of aspiration-penetration for the patient. Extracted features (or a reduced/weighted subset thereof) of acquired swallow-specific data can be compared with preset classification criteria to classify each data set as representative of a normal swallowing event or a potentially impaired swallowing event.

For example, the method 500 can optionally comprise a training/validation subroutine Step 516 in which a data set representative of multiple swallows is processed such that each swallow-specific data set ultimately experiences the preprocessing, feature extraction and feature reduction disclosed herein. A validation loop can be applied to the discriminant analysis-based classifier using a random hold-out cross-validation test and/or another cross-validation process. After all events have been classified and validated, output criteria may be generated for future classification without necessarily applying further validation to the classification criteria. Alternatively, routine validation may be implemented to either refine the statistical significance of classification criteria, or again as a measure to accommodate specific equipment and/or protocol changes (e.g. recalibration of specific equipment, for example, upon replacing accelerometer with same or different accelerometer type/model, changing operating conditions, new processing modules such as further preprocessing subroutines, artifact removal, additional feature extraction/reduction, etc.).

The classification can be used to determine and output which swallowing event represented a normal swallowing event as compared to a penetration, an aspiration, a swallowing safety impairment and/or an swallowing efficiency impairment at Step 514. In some embodiments, the swallowing event can be further classified as a safe event or an unsafe event.

For example, the processing module 106 and/or a device associated with the processing module 106 can comprise a display that identifies a swallow or an aspiration using images such as text, icons, colors, lights turned on and off, and the like. Alternatively or additionally, the processing module 106 and/or a device associated with the processing module 106 can comprise a speaker that identifies a swallow or an aspiration using auditory signals. The present disclosure is not limited to a specific embodiment of the output, and the output can be any means by which the classification of the swallowing event is identified to a user of the device 100, such as a clinician or a patient.

The output may then be utilized in screening/diagnosing the tested candidate and providing appropriate treatment, further testing, and/or proposed dietary or other related restrictions thereto until further assessment and/or treatment may be applied. For example, adjustments to feedings can be based on changing consistency or type of food and/or the size and/or frequency of mouthfuls being offered to the patient.

Alternative types of vibration sensors other than accelerometers can be used with appropriate modifications to be the sensor 102. For example, a sensor can measure displacement (e.g, a microphone), while the processing module 106 records displacement signals over time. As another example, a sensor can measure velocity, while the processing module 106 records velocity signals over time. Such signals can then be converted into acceleration signals and processed as disclosed herein and/or by other techniques of feature extraction and classification appropriate for the type of received signal.

Another aspect of the present disclosure is a method of treating dysphagia. The term “treat” includes both prophylactic or preventive treatment (that prevent and/or slow the development of dysphagia) and curative, therapeutic or disease-modifying treatment, including therapeutic measures that cure, slow down, lessen symptoms of, and/or halt progression of dysphagia; and treatment of patients at risk of dysphagia, for example patients having another disease or medical condition that increase their risk of dysphagia relative to a healthy individual of similar characteristics (age, gender, geographic location, and the like). The term does not necessarily imply that a subject is treated until total recovery. The term “treat” also refers to the maintenance and/or promotion of health in an individual not suffering from dysphagia but who may be susceptible to the development of dysphagia. The term “treat” also includes the potentiation or otherwise enhancement of one or more primary prophylactic or therapeutic measures. The term “treat” further includes the dietary management of dysphagia or the dietary management for prophylaxis or prevention of dysphagia. A treatment can be conducted by a patient, a clinician and/or any other individual or entity.

The method of treating dysphagia comprises using any embodiment of the device 100 disclosed herein and/or performing any embodiment of the method 500 disclosed herein. The method can further comprise adjusting a feeding administered to the patient based on the classification, for example by changing a consistency of the feeding, changing a type of food in the feeding, changing a size of a portion of the feeding administered to the patient, changing a frequency at which portions of the feeding are administered to the patient, or combinations thereof.

In an embodiment, the method prevents aspiration pneumonia from dysphagia. In an embodiment, the dysphagia is oral pharyngeal dysphagia associated with a condition selected from the group consisting of cancer, cancer chemotherapy, cancer radiotherapy, surgery for oral cancer, surgery for throat cancer, a stroke, a brain injury, a progressive neuromuscular disease, neurodegenerative diseases, an elderly age of the patient, and combinations thereof. As used herein, an “elderly” human is a person with a chronological age of 65 years or older.

### EXAMPLE

The following experimental example presents scientific data developing and supporting an embodiment of the automatic framework for detecting aspiration-penetration based on dual-axis (A-P and S-I) accelerometry signals that is disclosed herein.

Methodology

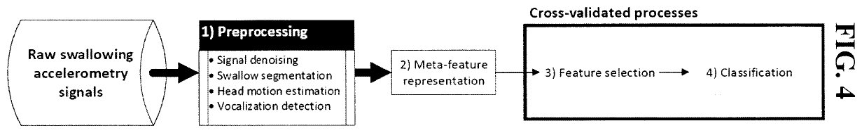

As shown in FIG. 4, the tested framework included 1) preprocessing and conditioning of accelerometry signals, 2) meta-feature based representation of the signals, 3) salient feature identification, and 4) classification of swallowing signals.

Specifically, the swallowing accelerometry data was subjected to a preprocessing stage to reduce artifacts, extract vocalizations segments (cough and speech), and estimate head motion component of the signals. In the preprocessing, the accelerometry signals were collected at a sampling frequency of 10 kHz because the majority of signal power in dual-axis swallowing accelerometry observations is concentrated below 100 Hz. The accelerometry signals were denoised via 10-level wavelet decomposition with Daubechies-8 mother wavelets and reconstructed with soft-thresholding.

The approximation and detail wavelet coefficients were also used to extract the signal component corresponding to head motion and to identify vocalization segments within the captured swallowing signals. Specifically, the approximation wavelet coefficients at level 10 were used to reconstruct the signal component containing frequencies less than 5 Hz that are reported to be the frequency content-characterizing head motion. To isolate the signal component with frequency content characterizing vocalization, everything except the detail wavelet coefficients corresponding to the frequency range 40-650 Hz (detail coefficients of level 5 to 8) was suppressed. Within the extracted vocalization component of the signal (40-650 Hz content), the active segments were identified via a peak search, and those segments with duration between 0.4 to 1 second were identified as vocalization segments.

The preprocessed signals (referred to as “bolus signals” hereafter) were also subjected to segmentation to identify regions of individual swallowing events using an energy-based segmentation approach. Although a particular accelerometry profile may be associated with distinct swallowing events, inter-personal differences in the amplitude range of these events may be present and can impede the inter-personal classification of bolus signals. To this end, channel-specific unity-based normalization (scaling the signals in the range [0, 1]) was applied on the bivariate bolus signals.

Then the preprocessed signals were represented in terms of time-frequency meta-features. Specifically, the preprocessed and normalized bivariate bolus signals (A-P and S-I channels) were converted to univariate signals via windowed inner-product of their A-P and S-I channels (window size of 750 samples with 90% overlap between successive windows). The resulting univariate bolus signals (referred to as “inner-product signals” hereafter) captured sequential interaction between the vibrations in A-P and S-I channels and highlighted concurrent active regions in the two channels, while suppressing channel-specific variabilities that might not be relevant to swallowing events. Furthermore, the windowed inner-product conversion further reduced the dimensionality of the accelerometry observations in half, resulting in univariate time-series observations. The choice of meta-features was informed by previous research on discriminative physiological signal processing, as well as visual and auditory inspections of class-specific swallowing accelerometry exemplars.

The resulting inner-product signals were then represented in terms of 34 meta-features to capture temporal and spectral characteristics of these signals. In addition, a set of meta-features were included to characterize head motions along the A-P and S-I channels. These features were computed using the estimated channel-specific head motions. Furthermore, for each channel-specific head-motion features, a corresponding relative feature, computed as the ratio of that feature in the A-P channel to the one measured in the S-I channel, was also added as a meta-feature.

The meta-feature representation of bolus signals was then used as the input, along with respective labels, in the subsequent feature-selection and classification. The feature-selection step identified meta-features most salient to detecting swallowing aspiration-penetration.

To account for imbalanced binary samples (minority positive class) at the bolus-level, the posterior threshold (based on which a signal was classified in the Bayesian classification) was tuned using the training set in each cross-validation run to optimize sensitivity and specificity of the classifier. In particular, an ROC curve was formed by the posteriors of training samples, and a point along the curve was selected that maximized sensitivity of the classifier while maintaining a minimum of 60% specificity (Equation 1). The performance of the classifier was then evaluated based on whether it could yield a test sensitivity and specificity of minimum 80% and 60%, respectively.

max Sensitivity, s.t.: Specificity>60% (1)

As can be seen in Equation 4, the participant-level roll-up rules tend to produce a sensitive classifier, and therefore the corresponding bolus-level classifier should also be tuned to achieve desired participant-level sensitivity and specificity. To this end, an ROC curve for the participant level roll-up classifier was constructed by computing the participant-level sensitivity and specificity at all the bolus level classification thresholds. A bolus-level classification threshold was then selected to achieve desired participant-level sensitivity and specificity rates. Similar to the bolus-level classification, a threshold was selected to maximize participant-level sensitivity while maintaining a minimum of 60% specificity (Equation 5). The participant-level threshold-tuning is also done using the training sets in cross-validation runs.

Experimental Set-Up

A dual-axis accelerometer (ADXL327, Analog devices) was used to record the acceleration signals at the participant's neck (anterior to cricoid cartilage) in anterior-posterior and superior-inferior directions. The accelerometer had a measurement range of 2.5±g and a sensitivity of 420 mV/g. The recorded signals were filtered to suppress frequencies beyond 0.1 Hz and 3 kHz and then resampled at 10 kHz. The frequency content lower than 0.1 Hz correspond to DC components and whole-body sway and are irrelevant to the swallowing activities. The resulting signals were then logged for further analysis.

Eight centers were involved in collecting swallowing accelerometry data. In total 305 patients participated in the study. Participants were stroke or brain injury survivors or adults over the age of 50 years who were referred for videofluoroscopy swallowing assessment on the basis of clinical symptoms of swallowing difficulty. After providing written consent and a sensor calibration task based on humming sounds, the participants were asked to take a number of comfortable sips of water followed by barium-infused liquids of different consistencies (each 3 boluses), while seated in a VFSS suite and having the accelerometer sensor attached on their neck anterior to cricoid cartilage. Four barium-infused liquids were tested: thin liquid barium, nectar-thick liquid barium (mild consistency), honey-thick liquid barium (moderate consistency), and pudding-thick liquid barium (thick consistency). A data collection session stopped if the attending clinician detected serious swallowing difficulties in the first two sips of water.

The VFSS screenings were available for the barium-infused liquids and were used to diagnose swallowing anomalies by two experienced speech-language pathologists (blinded to patient identity) using the 8-point Penetration-Aspiration Scale (PAS). The 8-point PAS scores swallowing activities based on the severity of airway invasion between 1 (no food entry to airway) and 8 (foodstuff enters the airway and passes below the vocal folds without clearance). For automatic detection of aspiration-penetration, the annotations by the speech-language pathologists were mapped to a binary label of safe (normal airway protection and high penetration; PAS ≤3) and unsafe (deeper entry of material into the airway without clearance; PAS >3).

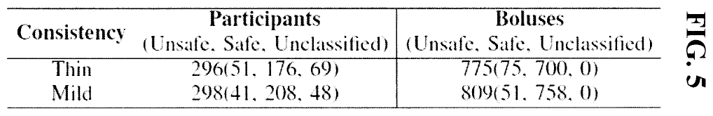

FIG. 5 shows the total number of participants and boluses in the study. The discriminative analysis disclosed herein was performed on thin and mild barium-infused liquids only, and the thick and moderate consistencies were excluded from the analysis in the current study due to severely imbalanced samples.

The classifier evaluation stage involved 1,000 runs of random hold-out cross-validation test, and the classification performance was reported in terms of mean(±standard deviation) sensitivity, specificity, and area under curve (AUC) across the cross-validation runs. Confidence intervals for these performance measures were also reported. The cross-validation runs were completely independent of one another, and a classifier trained in a cross-validation run was oblivious to the test set in that cross-validation run.

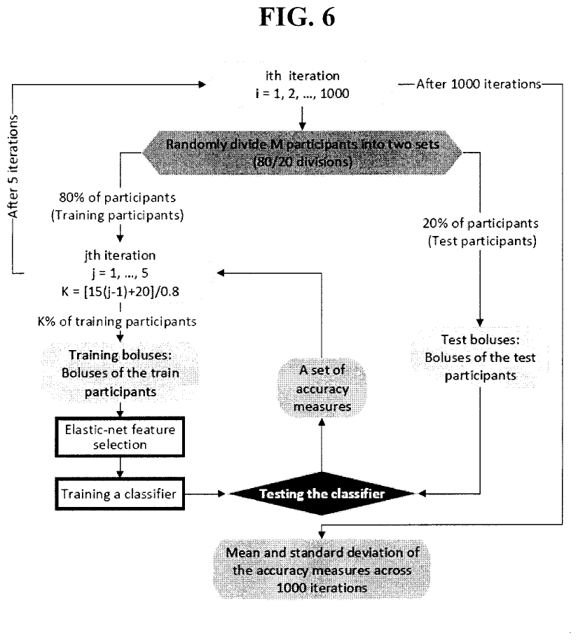

In each run of the hold-out cross-validation, the entire dataset was randomly divided into training participants (80% of participants) and test participants (20% of participants). A classifier was designed using boluses of the training participants and tested using boluses of the test participants in that cross-validation run. This process was repeated 1,000 times. FIG. 6 shows a schematic of the cross-validated classification experiment.

Furthermore, the effect on the classifier performance of having incrementally more training data, but the same test burden, was evaluated within the cross-validation test. The increment in the number of training participants was implemented in five iterations within each cross-validation run. In each cross-validation run, boluses from 25%, 44%, 62.5%, 81% and 100% of the training participants (80% of the total participants in the dataset) were used as training boluses in iterations 1 to 5, respectively. After 1,000 runs of the cross-validation were completed, summary performance measures were reported for each training set size (five different sizes; five iteration within each cross-validation run).

To set the parameters of the elastic net feature-selection (α and λ in Equation 3) in each cross-validation run, a two-dimensional internal cross-validation was conducted using only the training set in that run. A set of α values in the range of 0:05:0:05:1 and 100 values of λ were tested and the pair of α and λ with the minimum 10-fold cross-validation deviance (minus twice the log-likelihood on the test data in the internal cross-validation) was selected. To address the imbalance in the datasets (FIG. 5), minority positive samples (unsafe boluses) were weighted higher than negative samples at w=(number of negative samples)/(number of positive samples). Thus the logistic regression was penalized more for misclassifying positive samples. In a cross-validation run, the elastic net feature-selection was performed using the training set only and the selected features were then used to represent both training and test sets in that cross-validation run.

Results

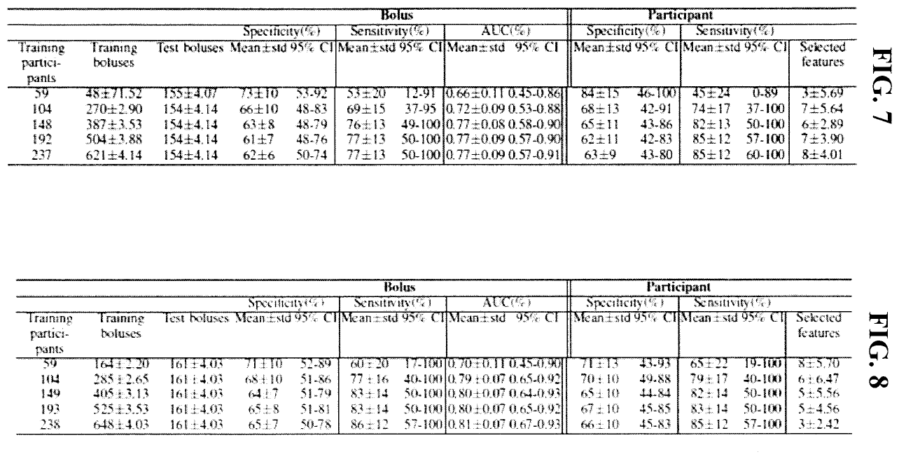

FIGS. 7 and 8 show the classification rates after 1,000 runs of hold-out cross-validation. In this cross-validation runs, feature-selection and classifier threshold-tuning were also done using training sets only. Some participants did not have a complete set of three boluses for thin and mild consistencies. Therefore, the number of test boluses from 20% test participants varied between cross-validation runs (1,000 runs); hence, standard deviation for the number of test boluses was included in FIGS. 7 and 8. As can be seen in these figures, an AUC of 0.8 was achieved in both thin and mild consistencies, and the classification rates improved with the increase in the number of training participants.

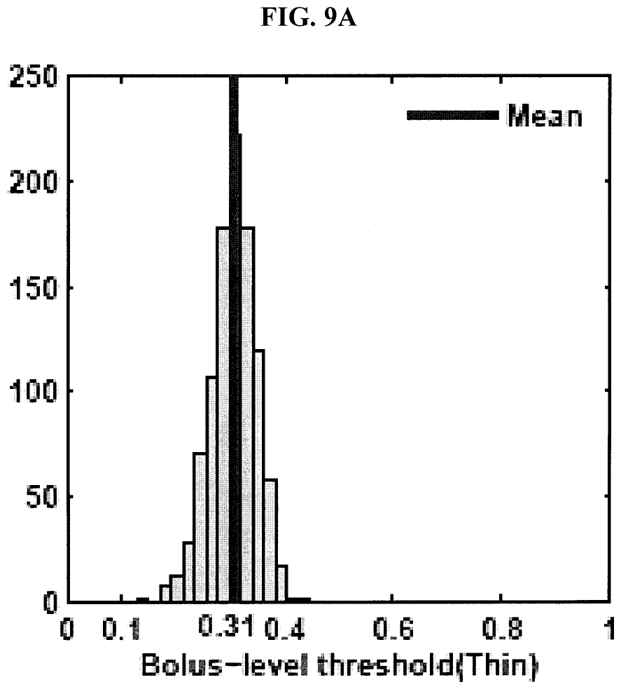

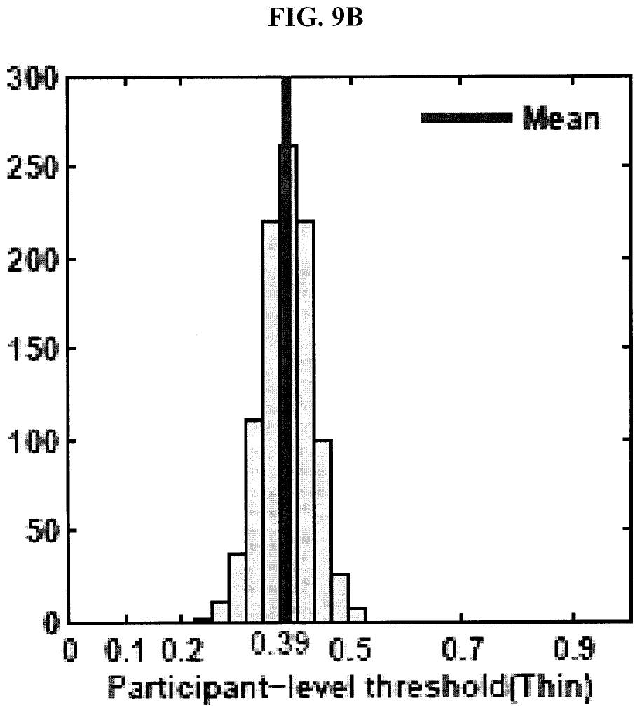

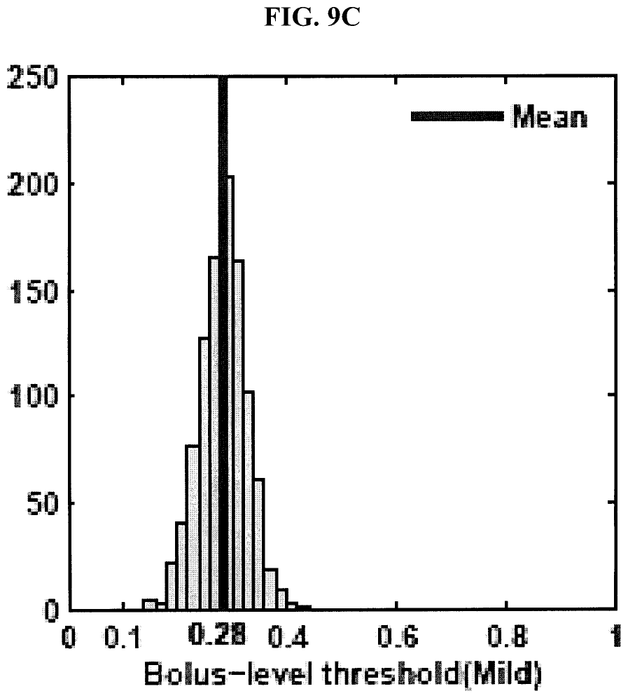

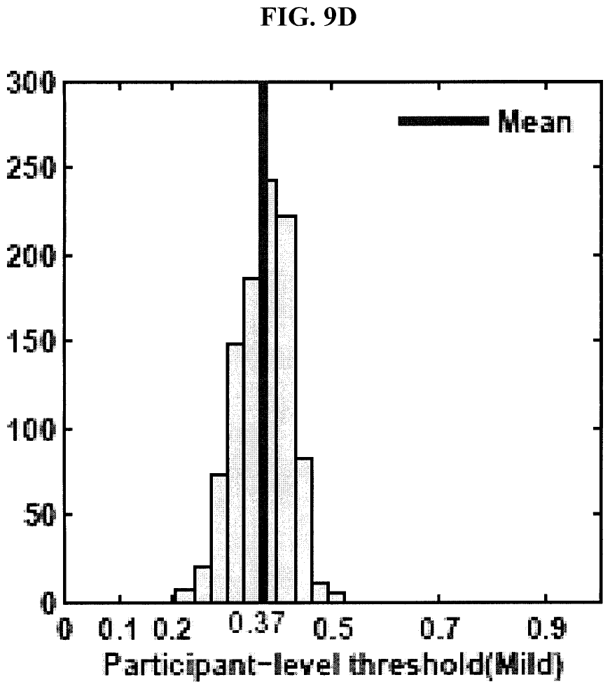

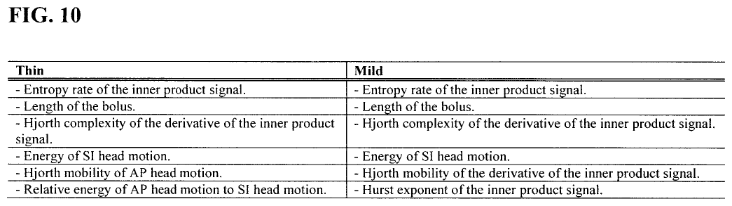

FIG. 10 shows the top six features identified as most salient for discriminating unsafe from safe boluses in thin and mild consistencies. The top six features were reported as the average number of features selected in the 1,000 runs of the cross-validation test for both thin and mild consistencies (last column in FIGS. 7 and 8). FIGS. 9A-D show the distributions of tuned classifier thresholds at bolus- and participant-levels in the 1,000 runs of the cross-validation test. As can be seen in FIGS. 9A-D, the bolus- and participant-level thresholds were different because the roll-up participant classifier was a sensitive one and therefore the corresponding bolus-level classifier was tuned separately to achieve the desired participant-level sensitivity and specificity.

The proposed classification framework achieved high bolus- and participant-level sensitivity and specificity rates in both thin and mild consistencies. The resulting classification performance surpasses previous reports on automatic detection of swallowing aspiration-penetration. Furthermore, the dataset used in the current study contains a larger number of participants (FIG. 5) than previous studies and is collected at 8 different sites. Therefore, the present dataset also contained a large amount of between-site and attending clinician variabilities, which made it challenging.

The performance of the automatic classification framework also exceeded clinical non-instrumental assessments (e.g., a detailed orofacial examination, voice assessment), which are generally subjective in assessment.

In general, the confidence intervals for specificity rates are narrower than those of sensitivity rates in FIGS. 7 and 8, which indicate more consistent specificity rates across the cross-validation runs. This is due in part to minority positive class in the cross-validation experiments. As a result, there were cross-validation runs where the training set contained a very small number of positive boluses (the boluses were divided at the participant-level and therefore the cross-validation divisions were not stratified at the bolus-level). Nevertheless, the results of this study were further proof that swallowing accelerometry signals provide discriminative information for detecting aspiration-penetration events.

In this study, classification rates improved with the increase in the training set size (FIGS. 7 and 8). Five one-way repeated measure ANOVA were conducted to explore the effect of training set size on classification rates. In these ANOVA tests, the independent variable was the number of training participants (five levels; first column of FIGS. 7 and 8), and the dependent variable was one of the following: 1) bolus-level sensitivity, 2) bolus-level specificity, 3) bolus-level AUC, 4) participant-level sensitivity, and 5) participant-level specificity. The effect of training set size on bolus- and participant-level rates was found significant (p<0.001) with partial η²>0.08 in all the cases, which indicates a large effect. Furthermore, a post-hoc pair-wise comparison with Bonferroni correction indicated significant difference between pairs of subsequent training sizes. Therefore, the increase in training set size significantly improved the classification rates in both thin and mild cases.

Classification permutation tests were also conducted to evaluate the null hypothesis that the achieved classification performance (FIGS. 7 and 8) was obtained by chance, only because during the training phase, the classifier identified a pattern that is random. The alternative hypothesis was that the proposed classifier captured the class structure and that there was a significant statistical connection between the identified salient meta-features and class labels. Random permutations of the dataset (1,000 times) were obtained by randomizing bolus- and participant-labels and running the classification framework. The permutation test resulted in an average AUC of 0.5 with p<0.01; hence indicating that the proposed classifier and identified meta-features are significant in discriminating between safe and unsafe boluses.

The discriminative framework is not limited to the thin and mild consistencies and can be readily used to design a classifier for detecting aspiration-penetration in thicker consistencies. In general, the swallowing aspiration-penetration is more prevalent in thinner consistencies, and the lack of sufficient positive boluses in thicker consistencies impedes discriminative analysis of corresponding swallowing signals for the automatic detection of abnormalities. Further, due to physiological differences between swallowing processes during the intake of thicker consistencies and thinner ones, a classifier trained on the latter can not be used to detect swallowing anomalies in thicker consistencies.

These consistency-specific swallowing characteristics are not relevant to detection of swallowing anomalies, and therefore any cross-consistency discriminative analysis should account for such between-consistency differences. In this study, a separate classifier was proposed and tested for each consistency.

As can be seen in FIG. 10, the top four identified features for the thin and mild consistencies are the same. Besides the bolus signal length, the selected features measure temporal and spectral complexity of the bolus signals. Unsafe boluses are generally longer in length and are characterized with multiple subswallows followed by coughs and throat clearing, all of which contribute to longer length and larger complexity of these boluses as compared to the safe ones. Entropy rate is a measure of regularity of the signal and quantifies sequential dependencies. The Hjorth mobility parameter of the A-P head motion is an estimate of the mean frequency of the head motion. Another identified feature is the Hjorth complexity that measures the spectral complexity of a signal through quantifying deviation from a sine wave. Hurst exponent is also found salient to discriminating between safe and unsafe mild boluses. Hurst exponent characterizes smoothness of a waveform and quantifies the long-range dependencies.

The bolus signals were represented in terms of the top most frequently selected features (FIG. 10) and a fixed set of bolus- and participant-level classifier thresholds was determined as the mean of the corresponding thresholds computed in the 1,000 runs of the hold-out cross-validation test (FIGS. 9A-D). Then, the cross-validation experiment was repeated with the fixed bolus representation and classification thresholds, and test bolus-level sensitivity rates of 87±13% and 89±11% and test specificity rates of 60±11% and 63±8% were obtained for the thin and mild boluses, respectively. Furthermore, test participant-level sensitivity rates of 89±12% and 89±12% and test participant-level specificity rates of 65±10% and 63±9% were achieved for thin and mild consistencies, respectively. The resulting classification rates indicate the discriminative power of the selected features and the classification framework for detecting aspiration-penetration problems.

As can be seen in FIG. 10, four out of the six identified features in thin and mild cases are identical. The performance of classifiers trained on thin boluses was tested in detecting aspiration-penetration in mild cases and had bolus-level sensitivity of 84±6% and specificity of 58±12% and participant-level sensitivity of 63±10% and specificity of 88±8%. These classification rates are similar to those reported in FIG. 8 and indicate the suitability of the identified features for detecting aspiration-penetration problem across thin and mild consistencies and that a classifier trained on one consistency can readily be used to detect problems in the other. Previous studies reported physiological similarities between swallowing of thin liquid (thin) and nectar-thick liquid (mild). In the present study, the identified salient features captured these similarities and were shown to discriminate aspiration-penetration in one consistency using a classifier trained on the other consistency.

### CONCLUSION

This study exploited swallowing accelerometry as an alternative non-invasive approach to videofluoroscopy for observing and diagnosis of swallowing aspiration-penetration. A dual-axis accelerometer was placed on the patient's neck to measure epidermal vibrations in the superior-inferior and anterior-posterior directions. The acquired accelerometry signals were then represented in terms of time-frequency meta-features, and the most discriminative of them were identified using the elastic net feature-selection. The identified features were then used to design a linear discriminant classifier to detect unsafe boluses (boluses with airway invasion at or below true vocal folds) and participants. The performance of the classifier was evaluated using 1,000 runs of hold-out cross-validation.

In each run, the training and test sets division was done at the participant-level. A bolus- and participant-level sensitivity >80% and specificity >60% were achieved for thin and mild consistencies in datasets of 296 and 298 participants, respectively. Furthermore, the classifier performance showed a significant improvement by having incrementally more training participants, but the same test burden.

The features most salient for discriminating aspiration-penetration were identified as features capturing the duration of activity in bolus accelerometry signals, along with those characterizing the temporal and spectral complexities of the signals. Additionally, similar salient features were identified for thin and mild consistencies, which indicated that the identified features are consistency-independent. To this end and using the identified salient features, the present study showed the reliability of the proposed classifier framework trained on thin swallowing samples to detect aspiration-penetration in mild samples. The results of the present study demonstrated the suitability of swallowing accelerometry signals for accurate detection of aspiration-penetration. An accurate detection of aspiration-penetration can help implementing more effective intervention, which in turn can reduce the dire health risks associated with aspiration-penetration.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method to classify a swallow, the method comprising:
receiving, on a processing module, dual-axis accelerometry signals obtained during the swallow by a sensor positioned externally on an anterior-posterior (A-P) axis and a superior-inferior axis (S-I) of the throat of a subject;
representing the dual-axis accelerometry signals as meta-features, the processing module performs the representing;
identifying a subset of the meta-features using regularized binomial logistic regression with elastic net penalty, the processing module performs the identifying; and
using the subset of the meta-features for determination of a linear discriminant classifier, the processing module performs the determination.

2. The method of claim 1, wherein the meta-features comprise time-frequency characteristics of the accelerometry signals.

3. The method of claim 1, wherein the meta-features comprise one or more channel-specific head-motion features.

4. The method of claim 3, wherein the meta-features comprise, for each of the one or more channel-specific head-motion features, a ratio of the channel-specific head-motion feature for the A-P axis to the corresponding channel-specific head-motion feature for the S-I axis.

5. The method of claim 1 comprising tuning the linear discriminant classifier by performing cross-validation to identify salient meta-features by regularized binomial logistic regression with elastic net penalty and to optimize at least one of sensitivity or specificity of the linear discriminant classifier.

6. The method of claim 5, wherein the linear discriminant classifier comprises a bolus-level threshold and a participant-level threshold, and the tuning of the linear discriminant classifier comprises tuning the bolus-level threshold and the participant-level threshold separately from each other.

7. The method of claim 1 comprising converting the dual-axis accelerometry signals from bivariate bolus signals to univariate bolus signals which are represented as a set of meta-features.

8. The method of claim 1 further comprising:
receiving, on the processing module, a set of bolus accelerometry signals;
applying the linear discriminant classifier to the set of bolus accelerometry signals; and
providing on the processing module or a device operatively connected to the processing module an indication whether the set of bolus accelerometry signals comprises an aspiration-penetration, the indication based on the applying of the linear discriminant classifier to the set of meta-features representing the bolus accelerometry signals.

9. A method to classify a swallow, the method comprising:
receiving, on a processing module, dual-axis accelerometry signals obtained during the swallow by a sensor positioned externally on an anterior-posterior (A-P) axis and a superior-inferior axis (S-I) of the throat of a subject;
representing the dual-axis accelerometry signals as meta-features, the processing module performs the representing;
comparing the salient meta-features, identified by regularized binomial logistic regression with elastic net penalty performed in a known training data set, with a preset linear discriminant classifier constructed on the salient time and frequency meta-features in a known training data set, the processing module performs the comparing; and
classifying the swallow as a normal swallow or an aspiration-penetration, the processing module performs the classifying based on the comparing.

10. The method of claim 9, wherein the meta-features comprise time-frequency characteristics of the accelerometry signals.

11. The method of claim 9, wherein the meta-features comprise one or more channel-specific head-motion features.

12. The method of claim 11, wherein the meta-features comprise, for each of the one or more channel-specific head-motion features, a ratio of the channel-specific head-motion feature for the A-P axis to the corresponding channel-specific head-motion feature for the S-I axis.

13. The method of claim 9 comprising tuning the linear discriminant classifier by performing cross-validation to identify salient meta-features by regularized binomial logistic regression with elastic net penalty and to optimize at least one of sensitivity or specificity of the linear discriminant classifier.

14. An apparatus for quantifying swallowing function, the apparatus comprising:
a sensor configured to be positioned on the throat of a patient and acquire vibrational data representing swallowing activity and associated with an anterior-posterior axis and a superior-inferior axis; and
a processing module operatively connected to the sensor and configured to (i) represent the vibrational data as salient meta-features identified by regularized binomial logistic regression with elastic net penalty performed on time and frequency meta-features in a known training data set, (ii) compare the salient meta-features with a preset linear discriminant classifier constructed using the time and frequency meta-features in the known training data set; and (iii) classify the swallow as a normal swallow or an aspiration-penetration, based on comparison of the salient meta-features with the preset linear discriminant classifier.

15. The apparatus of claim 14 comprising an output component selected from a display, a speaker, and a combination thereof, the processing module configured to use the output component to indicate the classification of the swallow visually and/or audibly.

16. The apparatus of claim 14, wherein the processing module is operatively connected to the sensor by at least one of a wired connection or a wireless connection.

17-18. (canceled)
